(19) United States
(12) Patent Application Publication
(10) Pub. No.: US 2023/0330315 A1
(43) Pub. Date: Oct. 19, 2023

(54) APPARATUS FOR EXTRACORPOREAL BLOOD TREATMENT AND METHOD FOR ADMINISTERING NUTRITIONAL PRODUCTS IN AN APPARATUS FOR EXTRACORPOREAL BLOOD TREATMENT

(71) Applicant: GAMBRO LUNDIA AB, Lund (SE)

(72) Inventor: Paolo Rovatti, Finale Emilia, (MO) (IT)

(21) Appl. No.: 17/914,924

(22) PCT Filed: Apr. 9, 2021
(86) PCT No.: PCT/EP2021/059331

§ 371 Date: Sep. 27, 2022

(30) Foreign Application Priority Data
Apr. 9, 2020 (EP) 20168991.6

(51) Int. Cl. A61M 1/34; A61M 1/16

(52) U.S. Cl. CPC A61M 1/3424; A61M 1/1694; A61M 1/3403

(57) ABSTRACT

An apparatus for extracorporeal blood treatment for chronic therapy comprises a nutritional bag (33) containing a nutritional solution, a weighing device (36) configured to determine a weight (W) of the nutritional bag (33), a nutritional line (34) for infusing the nutritional solution into either a blood return line (7) or into the patient vascular system. An ultrafiltration device (30) is configured to achieve a fluid removal from the patient through a semi- permeable membrane (5) of a filtration unit (2) and a sensor (31, 32) is configured to determine an ultrafiltration rate (UFR). A control unit (100) connected to the ultrafiltration device (30), to the weighing device (33) and to the sensor (31, 32) is programmed for receiving a patient prescription and for controlling the ultrafiltration device (30) to achieve the patient prescription based on the weight (W) and on ultrafiltration rate (UFR).

### DESCRIPTION

### TECHNICAL FIELD

The present invention relates to an apparatus for extracorporeal blood treatment and to a method for administering nutritional products in an apparatus for extracorporeal blood treatment.

Extracorporeal blood treatment involves removing blood from a patient, treating the blood externally to the patient, and returning the treated blood to the patient. Extracorporeal blood treatment is typically used to extract undesirable matter or molecules from the patient’s blood and add desirable matter or molecules to the blood. Extracorporeal blood treatment is used with patients unable to effectively remove matter from their blood, such as when a patient has suffered temporary or permanent kidney failure. These patients and other patients may undergo extracorporeal blood treatment to add or remove matter to their blood, to maintain an acid/base balance or to remove excess body fluids, or to perform extracorporeal gas exchange processes, for example.

In particular, the invention relates to chronic (long-term) hemodialysis (HD) therapy systems used to treat patients with chronic kidney failure. HD therapy is usually performed several times a week (e.g. 3 times a week) and each treatment has an average duration of some hours (e.g. 3.5 - 4.5 hours). During the therapy, the patient has two accesses (arterial/vein) where blood flows in/out from the body, wherein the average blood flow is usually between 250 and 400 ml/min.

### BACKGROUND OF THE INVENTION

The apparatuses for chronic (long-term) hemodialysis (HD) therapy comprise a device for online preparation of the dialysis liquid and/or replacement liquid. The device comprises one, two or more containers of concentrate located on respective injection lines which are predisposed to supply substances, such as electrolytes, buffer agents or others, towards a preparation line connected to a water source and located upstream a dialysis line. The concentrate containers may comprise concentrates in the liquid state or solid state, for example powder.

In patients receiving chronic hemodialysis, the National Kidney Foundation currently recommends a daily protein intake of 1.2 g/kg or more in patients undergoing hemodialysis. When malnutrition is present, a stepwise approach to treatment is used, beginning with dietary counseling and diet modifications, followed by oral nutritional supplements, and then by enteral nutrition supplements (usually consisting of a mixture of amino acids, glucose, and lipids) or parenteral nutritional supplements if needed.

In hemodialysis, it is known to administer IntraDialytic Parenteral Nutrition (IDPN) infusion e.g., through a venous access into the patient, typically, some minutes after dialysis has begun, and continued throughout the remainder of a dialysis session.

This is done by means of dedicated devices, such a bag, a dedicated line and a pump which are operated manually. This requires additional nurse workload.

In addition, the dialysis therapy parameters are corrected manually on the base of the operator experience, measurements or knowledge of the additional fluid/weight it is infused in this way. The manual correction of the dialysis therapy parameters does not allow to calculate in accurate manner the mass balance of the patient.

This is also a cumbersome and invasive solution and it is not usually well accepted by the patients/users.

An aim of the present invention is to provide for an apparatus for extracorporeal blood treatment that alleviates or minimizes or remedy the above-mentioned drawbacks.

It is an aim of the present description to provide an extracorporeal blood treatment apparatus and a method for administering nutritional products in an apparatus for extracorporeal blood treatment which are able to take into account the administered nutritional products to properly manage the mass balance of the patient during treatment.

It is an aim of the present invention to better control the administration of the nutritional products during therapy.

It is a further aim of the present description to control administration of the nutritional products through the same apparatus for extracorporeal blood treatment.

It is a further aim of the present description to reduce the workload of nurses.

It is a further aim of the present description to reduce complexity of devices dedicated to extracorporeal blood treatment with simultaneous administration of nutritional products.

It is a further aim to improve the comfort of patients undergoing treatment.

Document US5776345 is also known. This document discloses machine for acute treatment provided with a blood circuit to which a plurality of infusion lines may infuse fluids. A dialysis fluid container, a collection fluid container, a replacement fluid container and an anticoagulant container are connected to scales which weigh the contents. The replacement fluid adds material to the blood in order to adjust the pH of the blood, to add nutrients to the blood or to add fluid to the blood. Document US2012143116 discloses a renal failure therapy system including a blood pump, a dialysis fluid pump, a filtrate pump, one or more external infusion pumps, and a control unit configured to synchronize operation of the infusion pump to the blood pump and the filtrate pump. External pumps are fluidly connected to the blood return line. Each of the external pumps controllers pulls fluid from supplies which can include any suitable or desirable fluid, such as heparin, citrate, an electrolyte solution, an intravenous fluid, an antibiotic, a vasoactive drug, a total parenteral nutrition solution, an enteral nutrition solution fluid. The equation used to determine the calculated filtrate actual pump rate is based on filtrate pump rate=total input rate-total external output rate+prescribed net loss rate.

Document EP2644215 discloses an intensive care apparatus for extracorporeal treatment of blood with a filtration unit, a blood circuit, a pre and/or post-dilution fluid line connected to the blood circuit, and a dialysis circuit. Pumps act on the fluid lines for regulating the flow of fluid. A control unit is configured to periodically calculate a new value for the patient fluid removal rate to be imposed on an ultrafiltration actuator in order to keep a predefined patient fluid removal rate across a reference time interval irrespective of machine down times. The apparatus presents an infusion line connected with the blood withdrawal line upstream the blood pump and with an infusion fluid container, which contain a drug, or a regional anticoagulant, or a nutrients solution or other. This infusion line is referred to as pre-blood pump infusion line.

### SUMMARY

An apparatus according to one or more of the appended claims, taken singly or in any combination, attains at least one of the above-indicated aims.

An apparatus and a method according to aspects of the invention and capable of achieving one or more of the above objects are here below described.

In a 1^{st} independent aspect there is provided an apparatus for extracorporeal blood treatment for chronic (long term) therapy, comprising:
a filtration unit (2) having a primary chamber (3) and a secondary chamber (4) separated by a semi-permeable membrane (5);
a blood circuit coupled to the filtration unit (2) and comprising a blood withdrawal line (6) connected to an inlet (3a) of the primary chamber (3), a blood return line (7) connected to an outlet (3b) of the primary chamber (3), said blood withdrawal line (6) and blood return line (7) being configured for connection to a patient cardiovascular system;
a blood pump (10) configured to be coupled to a pump section of the blood circuit;
a dialysis circuit comprising a dialysis supply line (11), for a fresh dialysis fluid, connected to an inlet (4a) of the secondary chamber (4) and a dialysis effluent line (12), for a spent dialysis fluid, connected to an outlet (4b) of the secondary chamber (4);
a preparation device (13) for preparing the fresh dialysis fluid, wherein the preparation device (13) is connected to the dialysis supply line (11) and comprises a regulating device (14) for regulating the composition of the fresh dialysis fluid;
at least one nutritional bag (33) containing a nutritional solution;
a nutritional line (34) having a first end in fluid communication with the nutritional bag (33) and a second end for infusing the nutritional solution into either the blood return line (7) or directly into the patient vascular system;
at least one sensing element (36) configured to provide a first signal related to an actual flow rate in the nutritional line;
an ultrafiltration device (30) configured to achieve a fluid removal from the patient through the semi-permeable membrane (5);
at least one sensor (31, 32) configured to provide a second signal related to an ultrafiltration rate (UFR);
a control unit (100) connected at least to the ultrafiltration device (30), to the sensing element (36)and to the at least one sensor (31, 32) and programmed for:
   receiving a patient prescription including at least one of:
      a total patient weight loss (WL_{target}) to be achieved at the end of the blood treatment and a total treatment time (T);
      a desired net ultrafiltration rate (nUFR_{target});
   collecting from the sensing element (36) the first signal and determining a first parameter (W) related to the actual flow rate in the nutritional line (34);
   collecting from the at least one sensor (31, 32) the second signal and determining a second parameter (FR) related to the ultrafiltration rate (UFR);
   controlling the ultrafiltration device (30) to achieve the patient prescription based on the first parameter (W) and on the second parameter (FR).

In a 2^{nd} independent aspect there is provided a method for administering nutritional products in an apparatus for extracorporeal blood treatment, wherein the method comprises:
delivering, during an extracorporeal blood treatment of a chronic (long term) therapy of a patient, a nutritional solution from at least one nutritional bag into either a blood return line of an apparatus for extracorporeal blood treatment or directly into the patient vascular system;
collecting from a sensing element, in particular a weighing device, a first signal and determining based on the first signal a first parameter (W) related to an actual flow rate in the nutritional line, in particular a weight of the at least one nutritional bag;
collecting from at least one sensor a second signal and determining based on the second signal a second parameter (FR) related to an ultrafiltration rate (UFR) (i.e. difference between spent dialysis fluid exiting a secondary chamber of a filtration unit of the apparatus for extracorporeal blood treatment and prepared fresh dialysis fluid routed to a primary chamber of the filtration unit and/or infused into a blood circuit of said apparatus);
controlling an ultrafiltration device to achieve a patient prescription based on the first parameter (W) and on the second parameter (FR); wherein the patient prescription includes at least one of:
   a total patient weight loss (WL_{target}) to be achieved at the end of the blood treatment and a total treatment time (T);
   a desired net ultrafiltration rate (nUFR_{target}).

In a further aspect according to the previous aspects, the at least one sensing element (36) is a weighing device, in particular a weight scale, configured to provide a weight of the at least one nutritional bag (33), in particular wherein the control unit (100) is configured to receive a weight signal from the weighing device and to determine the first parameter (W) based on the weight variation over time of the nutritional bag, the first parameter being optionally the actual flow rate in the nutritional line.

The desired net ultrafiltration rate (nUFR_{target}) may be a constant value during blood treatment or may change over time during blood treatment according to a function nUFR_{target}(t).

The sensing element is in particular a scale collecting the weight of the nutritional bag over time.

In another aspect according to any one of the previous aspects, the patient prescription includes at least one of:
ii. a total patient weight loss (WL_{target}) to be achieved at the end of the blood treatment and a total treatment time (T);
iii. an ultrafiltration rate (UFR) and a total treatment time (T);
iv. a total patient weight loss (WL_{target}) to be achieved at the end of the blood treatment and an ultrafiltration rate (UFR);
v. a desired net ultrafiltration rate (nUFR_{target}) and a nutritional flow rate (or a nutritional total weight to be infused during the treatment) together with a total treatment time (T) or together with a total patient weight loss (WL_{target}).

Notably, since the treatment is a chronic treatment, one of the prescription target to achieve is the total patient weight loss (WL_{target}) at the end of the treatment, namely at the end of the total treatment time (T). In order to set up the machine the operator input data sufficient to calculate determine or acquire such values. On one side, the usual prescription includes total patient weight loss (WL_{target}) and total treatment time (T); this may be provided by the e.g., physician or included in the patient prescription card. However, as an alternative, the total treatment time (T) may be provided with the ultrafiltration rate (UFR). These two values allow to calculate the total patient weight loss (WL_{target}). Indeed, the three values are linked by the following relation:

$$U F R = \frac{W {L}_{t a r g e t}}{T}$$

In other terms, availability of two out of three terms allows to determine the third one (e.g., UFR and WL_{target} allow to determine total treatment time).

Whenever, a further infusion (nutrition infusion) is added and is not sensed with the sensor (31, 32) configured to provide the second signal related to an ultrafiltration rate (UFR), the net ultrafiltration rate has to be increased of a quantity identical to the nutrition infusion rate to achieve the total patient weight loss (WL_{target}) at the end of the treatment. Therefore, to achieve the total patient weight loss (WL_{target}), the prescription may include the desired net ultrafiltration rate (nUFR_{target}) and either a nutritional flow rate or a nutritional total weight to be infused during the treatment. With these two values and either the total treatment time (T) or the total patient weight loss (WL_{target}), again the apparatus may control the dialysis treatment reaching the desired and prescribed total weight loss in the desired and prescribed total treatment time.

In a 3^{rd} aspect according to aspect 1 or 2, a feeding rate (Qₙᵤₜᵣ) of the nutritional solution is delivered through the nutritional line; optionally an infusion pump is coupled to the nutritional line to deliver the feeding rate (Qₙᵤₜᵣ) of the nutritional solution through the nutritional line.

The infusion pump is particularly a peristaltic pump.

In a 4^{th} aspect according to any one of aspects 1 to 3, the control unit is connected to the infusion pump and is programmed for controlling the infusion pump and optionally for changing a feeding rate (Qₙᵤₜᵣ) of the nutritional solution.

In a 5^{th} aspect according to any one of aspects 1 to 4, the nutritional solution is delivered by commanding the infusion pump coupled to the nutritional line.

In a 5^{th} bis aspect according to any one of aspects 1 to 5, the extracorporeal blood treatment apparatus includes a main body, the infusion pump is attached to the main body, in particular is placed on a front panel of the main body.

In a 5^{th} ter aspect according to any one of aspects 1 to 5 bis, the nutritional line comprises a pump segment, a feeding tube segment connected to an inlet of the pump segment, and a delivery tube segment connected to an outlet of the pump segment.

In particular, the pump segment having a passage section for the nutritional solution larger than a passage section of the delivery tube segment and/or than a passage section of the feeding tube segment. In more detail, the passage section of the delivery tube segment is equal to the passage section of the feeding tube segment. Optionally, the nutritional line is a disposable line.

In a 5^{th} quarter aspect according to the previous aspect, the feeding tube segment and/or the delivery tube segment include/s a respective removable connector, in particular a Luer connector.

In more detail, the removable connector of the delivery tube segment being connectable to a corresponding counter connector on the blood return line, for example on a deareation chamber.

In a 5^{th} quinquies aspect according to any one of the previous two aspects, the nutritional line further comprises at least one rigid portion comprising two pump connectors for receiving opposite ends of the pump segment, a delivery tube segment connector for receiving one end of the delivery tube segment and a feeding tube segment connector for receiving one end of the feeding tube segment.

In a 5^{th} sexies aspect according to the previous aspect, the extracorporeal blood treatment apparatus includes a main body including a coupling device, the rigid body being configured to couple with the apparatus coupling device to position the nutritional line on the main body in specific arrangement with respect to the infusion pump, in particular the coupling device is placed on a front panel of the main body.

In a 6^{th} aspect according to any one of aspects 3 or 4 or 5, the method comprises or the control unit is programmed for: commanding the infusion pump to feed the nutritional solution according to a feeding rate target (Qₙᵤₜᵣ _{target}) during blood treatment; optionally the feeding rate target (Qₙᵤₜᵣ _{target}) may be a constant value during blood treatment or may change over time during blood treatment according to a function (Qₙᵤₜᵣ _{target}(t)).

In a 7^{th} aspect according to any one of aspects 1 to 6, the first parameter (W) is the weight (W(t)) of the nutritional bag at an instant of time or an actual feeding rate in the nutritional line.

In a 7^{th} bis aspect according to any one of aspects 1 to 7, the method comprises or the control unit is programmed for: controlling the infusion pump based on determining a feeding rate (Qₙᵤₜᵣ) based on the first parameter (W), comparing the feeding rate target (Q_{nutr target}) during blood treatment and the feeding rate (Qₙᵤₜᵣ) and reducing a difference between the feeding rate target (Qₙᵤₜᵣ _{target}) and the feeding rate (Qₙᵤₜᵣ).

In an 8^{th} aspect according to any one of aspects 1 to 7, the second parameter (FR) is the ultrafiltration rate (UFR).

In a 9^{th} aspect according to aspects 7 and 8, a net ultrafiltration rate (nUFR) is calculated by calculating a feeding rate (Qₙᵤₜᵣ) of the nutritional solution from the weight (W(t)) of the nutritional bag (33), in particular the weight variation over time, and by subtracting the feeding rate (Qₙᵤₜᵣ) from the ultrafiltration rate (UFR).

In a 9^{th} bis aspect according to aspects 7 and 8, a net ultrafiltration rate (nUFR) is calculated by subtracting the feeding rate (Qₙᵤₜᵣ) from the ultrafiltration rate (UFR).

As above explained, usually in an apparatus for extracorporeal blood treatment for chronic therapy with the fresh dialysis fluid (for feeding the filtration unit and/or for infusion into the extracorporeal blood circuit) on-line prepared, the sensor (31, 32) for providing the second signal related to an ultrafiltration rate (UFR), allows the control unit to calculate the ultrafiltration rate (UFR). In chronic machine of the state of the art, all dialysis and replacement fluid flows are taken into account by the UFR since the sensor/s senses all fluids fed and removed from blood. In the current embodiments in accordance with the present aspects and claims, situation is different. Indeed, the sensor (31, 32) for providing the second signal related to an ultrafiltration rate (UFR) are not intended to and does not provide a second signal taking into account an infused nutritional solution amount.

In a 9^{th} ter aspect according to any one of the previous aspects, the sensor (31, 32) for providing the second signal related to an ultrafiltration rate (UFR) takes into account a net amount/flow rate of the fresh dialysis fluid prepared by the preparation device (13), irrespective of the fresh dialysis fluid being fed to the filtration unit, removed from the filtration unit and infused into the blood circuit.

In a 9^{th} quater aspect according to any one of the previous aspects, the sensor (31, 32) for providing the second signal related to an ultrafiltration rate (UFR) does not take into account amount/flow rate of the nutritional solution.

In a 9^{th} quinquies aspect according to any one of the previous aspects, the sensor (31, 32) for providing the second signal related to an ultrafiltration rate (UFR) senses one or more of:
a difference between an amount or a flow rate of fresh dialysis fluid prepared by the preparation device (13) and entering into the filtration unit (2) and a spent dialysis fluid exiting the filtration unit (2);
a difference between an amount or a flow rate of fresh dialysis fluid prepared by the preparation device (13) and infused into the blood circuit and a spent dialysis fluid exiting the filtration unit (2); and
a difference between an amount or a flow rate of fresh dialysis fluid prepared by the preparation device (13) and entering both into the filtration unit (2) and into the blood circuit and a spent dialysis fluid exiting the filtration unit (2).

Therefore the sensor may work in any one of the HD, HF and HDF modes as above indicated.

In a 10^{th} aspect according to any one of previous aspects 9, the method comprises or the control unit is programmed for: controlling the ultrafiltration device so that the net ultrafiltration rate (nUFR) matches the desired net ultrafiltration rate (nUFR_{target}).

In a 11^{th} aspect according to previous aspects 7 and 8, a patient weight loss (WL(t)) at an instant of time is calculated by integrating the ultrafiltration rate (UFR) at that instant of time and by subtracting the weight (W(t)) of the nutritional bag at that instant of time from the integrated ultrafiltration rate (UFR).

In a 12^{th} aspect according to any one of the previous aspects, the method comprises or the control unit is programmed for:

controlling the ultrafiltration device so that the patient weight loss (WL) at the end of the blood treatment matches the total patient weight loss (WL_{target}) to be achieved at the end of the blood treatment (programmed, wanted or expected).

In a 13^{th} aspect according to any one of previous aspects 1 to 12, the method comprises or the control unit is programmed for:
receiving, as prescription input:
   a desired blood flow rate (Q_{b} _{target});
   a desired nutritional feeding rate target (Qₙᵤₜᵣ _{target}) or a total amount of the nutritional solution (Wₙᵤₜᵣ _{target}) administered at the end of the treatment time (T);
   the total patient weight loss (WL_{target}) and the total treatment time (T) or the desired net ultrafiltration rate (nUFR_{target});
commanding the blood pump to pump blood according to the desired blood flow rate (Q_{b target}) or to reach the total amount of the nutritional solution (Wₙᵤₜᵣ _{target}) administered at the end of the total treatment time (T);
commanding the infusion pump to deliver the nutritional solution according to the nutritional feeding rate target (Qₙᵤₜᵣ target);
commanding the ultrafiltration device to achieve the total patient weight loss (WL_{target}) or the desired net ultrafiltration rate (nUFR_{target}).

In a 13^{th} bis aspect according to any one of previous aspects, the method comprises or the control unit is programmed to receive, as prescription input, a total amount of the nutritional solution (Wₙᵤₜᵣ _{target}) to be administered within the end of the total treatment time (T), to determine a nutritional feeding rate target (Qₙᵤₜᵣ _{target}) to deliver the total amount of the nutritional solution (Wₙᵤₜᵣ _{target}) at the latest at an end of the total treatment time (T) and to command the infusion pump to deliver the nutritional solution according to the nutritional feeding rate target (Qₙᵤₜᵣ _{target}).

In a 14^{th} aspect according to any one of aspects 1 to 13, the control unit is programmed for calculating and/or storing data related to the nutritional solution administered during the extracorporeal blood treatment.

In a 15^{th} aspect according to previous aspect 14, said data related to the nutritional solution comprise at least one of: the weight of the at least one nutritional bag (W), a feeding rate (Qₙᵤₜᵣ) of the nutritional solution through the nutritional line, an amount of nutritional solution administered at an instant of time, a total amount of the nutritional solution to be administered, a composition of the nutritional solution.

In a 16^{th} aspect according to any one of aspects 14 or 15, wherein the method comprises or the control unit is programmed for: displaying on a display screen the data related to the nutritional solution.

In a 17^{th} aspect according to any one of aspects 1 to 16, the apparatus comprises a display screen connected to the control unit.

In an 18^{th} aspect according to any one of aspects 1 to 17, at least one weighing device is a weight scale.

In a 19^{th} aspect according to any one of aspects 1 to 18, the at least one nutritional bag is configured to be hanged on the at least one weighing device and/or the weighing device is configured to hang the nutritional bag.

In a 19^{th} bis aspect according to any one of aspects 1 to 5, the extracorporeal blood treatment apparatus includes a main body, the at least one weighing device is attached to the main body, in particular is placed on lateral portion of the main body.

In a 20th aspect according to any one of aspects 1 to 19, collecting the weight comprises: measuring the weight at instants of time; optionally with an acquisition frequency between 0.01 Hertz and 100 Hertz.

In a 21st bis aspect according to any one of the previous aspects, the apparatus comprises an infusion line (11′) branching from the dialysis supply line (11) to infuse fresh dialysis fluid into the blood circuit, the sensor (31, 32) configured to provide a second signal related to an ultrafiltration rate (UFR) being operative at least upstream a branch of the infusion line (11′) to take into account for the fresh dialysis fluid fed either or both to the filtration unit (2) through the dialysis supply line (11) or/and the blood circuit through the infusion line (11′).

In a 21st bis aspect according to the previous aspect, the infusion line (11′) is a branch infusion line receiving fresh dialysis fluid prepared by the preparation device (13).

In a 21^{st} aspect according to any one of aspects 1 to 20, the at least one sensor comprises at least one flowmeter placed on at least one of the dialysis supply line and the dialysis effluent line; wherein the second signal is a signal from said at least one flowmeter.

In a 21^{st} bis aspect according to any one of aspects 1 to 21, the at least one sensor comprises at least one differential flowmeter placed on the dialysis supply line and the dialysis effluent line to sense a differential flow between the dialysis supply line and the dialysis effluent line; wherein the second parameter is a signal from said at least one differential flowmeter.

In a 21st ter aspect according to any one of the previous aspects, the apparatus comprises an infusion line (11′) branching from the dialysis supply line (11) to infuse fresh dialysis fluid into the blood circuit, the differential flowmeter configured to provide a second signal related to an ultrafiltration rate (UFR) being operative at least upstream a branch of the infusion line (11′) to take into account for the fresh dialysis fluid fed either or both to the filtration unit (2) through the dialysis supply line (11) or/and the blood circuit through the infusion line (11′).

In a 21^{st} quater aspect according to any one of aspects 1 to 21, the at least one sensor comprises a flowmeter placed on the dialysis supply line and another flowmeter placed on the dialysis effluent line, in particular the control unit is configured to receive the signals from the two flowmeters to determine a differential flow between dialysis fluid supplied to the supply line and removed with the dialysis effluent line.

In a 21^{st} quinques aspect according to any one of the previous aspects, the apparatus comprises an infusion line (11′) branching from the dialysis supply line (11) to infuse fresh dialysis fluid into the blood circuit, the flowmeter on the dialysis supply line being operative at least upstream a branch of the infusion line (11′) to take into account for the fresh dialysis fluid fed either or both to the filtration unit (2) through the dialysis supply line (11) or/and the blood circuit through the infusion line (11′).

In a 22^{nd} aspect according to any one of aspects 1 to 20, the apparatus for extracorporeal blood treatment further comprises:
balance chambers operatively coupled to the dialysis circuit to precisely balance fresh dialysis fluid prepared by the preparation device with spent dialysis fluid exiting the filtration unit; and
a ultrafiltration line and pump to remove spent dialysis fluid from upstream the balance chamber in the dialysis effluent line; the at least one sensor sensing the amount of spent dialysis fluid removed by the ultrafiltration pump, wherein the second parameter is, e.g., a flow rate in the ultrafiltration line or a liquid volume removed through the ultrafiltration line.

In a 23^{rd} aspect according to any one of aspects 1 to 22, the ultrafiltration device comprises at least one dialysis pump coupled to the dialysis supply line and/or to the dialysis effluent line; optionally the at least one dialysis pump comprises a first dialysis pump coupled to the dialysis supply line and a second dialysis pump coupled to the dialysis effluent line.

In a 24^{th} aspect according to previous aspect 23, the at least one dialysis pump is a volumetric pump.

In a 25^{th} aspect according to previous aspect 24, the at least one dialysis pump is crossed by dialysis/effluent fluid.

In a 25^{th} bis aspect according to any one of the previous aspects, a first balance chamber operates on the dialysis supply line and a second balance chamber operates on the dialysis effluent line.

In a 26^{th} aspect according to any one of aspects 1 to 25, the infusion pump and/or the blood pump is/are peristaltic pumps.

In a 27^{th} aspect according to previous aspect 26, the infusion pump and/or the blood pump is/are coupled to tube section/s of the respective nutritional line or blood circuit.

In a 28^{th} aspect according to any one of aspects 1 to 27, the blood circuit and the filtration unit are disposable and are coupled in removable manner to a main body of the apparatus and to the blood pump.

In a 29^{th} aspect according to any one of aspects 1 to 28, the nutritional line is disposable and is coupled in removable manner to a main body of the apparatus and to the infusion pump.

In a 30^{th} aspect according to any one of aspects 1 to 29, the dialysis circuit is not-disposable; optionally the dialysis circuit is configured to be sterilized after each or a predetermined number of blood treatments.

In a 31^{st} aspect according to any one of aspects 1 to 30, the apparatus comprises a main body; wherein the infusion pump and the blood pump are supported by the main body; optionally a rotor of the infusion pump and a rotor of the blood pump are placed on a face of the main body.

In a 32^{nd} aspect according to previous aspect 31, a pump section of the blood circuit is coupled in removable manner to a rotor of the blood pump; a pump section of the nutritional line is coupled in removable manner to a rotor of the infusion pump.

In a 33^{rd} aspect according to aspects 31 or 32, the dialysis circuit is integrated in the main body; optionally the dialysis circuit is mounted fixed on the main body; optionally said at least one dialysis pump is mounted on the main body.

In a 34^{th} aspect according to any one of the previous aspects 31 to 33, the control unit is contained in or supported by the main body.

In a 34^{th} bis aspect according to any one of the previous aspects, the apparatus comprises a main body; wherein the main body includes a water inlet for feeding purified water to the preparation line.

In a 35^{th} aspect according to any one of the previous aspects 1 to 34, the apparatus comprises the nutritional solution contained in said at least one nutritional bag, wherein the nutritional solution is a parenteral nutritional solution

In a 36^{th} aspect according to any one of the previous aspects 1 to 35, the nutritional solution comprises a mixture of protein, carbohydrate and fat; optionally of amino-acids, glucose or dextrose and lipids.

In a 37^{th} aspect according to the previous aspect 36, the nutritional solution consists of a solution of amino-acids at a concentration of 10% - 20%, a solution of glucose or dextrose at a concentration of 40% - 70%, a solution of lipids at a concentration of 15% - 30%.

In a 38^{th} aspect according to any one of the previous aspects 1 to 37, a feeding rate (Qₙᵤₜᵣ) of the nutritional solution during blood treatment is between 50 ml/h and 500 ml/h, optionally between 100 ml/h and 200 ml/h, optionally of 150 ml/h.

In a 39^{th} aspect according to previous aspects to any one of the previous aspects 1 to 38, a total amount of the nutritional solution administered at the end of the blood treatment is between 100 ml and 1000 ml, optionally between 300 ml and 500 ml.

In a 40^{th} aspect according to any one of the previous aspects 1 to 39, the nutritional solution provides 150 - 200 kcal/h.

In a 41^{st} aspect according to any one of the previous aspects 1 to 40, a plurality of nutritional bags are provided, each nutritional bag containing a component of the nutritional solution; optionally a first bag contains a solution of amino-acids, a second bag contains a solution of glucose or dextrose, a third bag contains a solution of lipids.

In a 42^{nd} aspect according to the previous aspect 41, the plurality of nutritional bags are all connected to a common nutritional line or to the blood return line or directly to the patient vascular system.

In a 42^{nd} aspect according to any one of the previous aspects 1 to 41, a total treatment time of the extracorporeal blood treatment is between 0.5 to 10 hours, optionally between 3 hours and 5 hours.

In a 43^{rd} aspect according to any one of the previous aspects 1 to 42, the preparation device comprises a preparation line and at least one container, optionally a plurality of containers, of concentrate/s; wherein the container/s is/are located on respective injection line/s predisposed to supply substances to the preparation line; wherein the preparation line is connected to a liquid source, optionally a water source, optionally a reverse osmosis water plant.

In a 44^{th} aspect according to the previous aspect 43, the regulating device comprises at least one injection pump placed on the injection line/s.

In a 45^{th} aspect according to the previous aspects 43 or 44, the regulating device comprises at least one sensor placed on the injection line/s and/or on the preparation line.

In a 46^{th} aspect according to the previous aspect 45, said at least one sensor is configured to detect a flow rate or a concentration or a conductivity of the substances and/or of the fresh dialysis fluid through the injection line/s and/or on the preparation line.

In a 47^{th} aspect according to any one of the preceding aspects 1 to 46, at least one infusion line is connected to the blood circuit and to an infusion liquid source.

In a 48^{th} aspect according to any one of the preceding aspect 47, the at least one infusion line is connected to the dialysis supply line and the infusion liquid is the fresh dialysis liquid.

In a 49^{th} aspect according to any one of the preceding aspects 47 or 48, the at least one infusion line is connected to the blood return line.

Further characteristics of the present invention will better emerge from the detailed description that follows of some embodiments of the invention, illustrated by way of non-limiting examples in the accompanying figures.

### DESCRIPTION OF THE DRAWINGS

The description will now follow, with reference to the appended Figures, provided by way of non-limiting example, in which:

FIG. 1 shows a front view of an extracorporeal blood treatment apparatus according to the invention;

FIG. 1a shows a disposable nutritional line;

FIG. 2 schematically shows the extracorporeal blood treatment apparatus of FIG. 1;

FIG. 3 shows an enlarged portion of the extracorporeal blood treatment apparatus of FIG. 2;

FIG. 4 shows a portion according to a variant embodiment of the apparatus of FIGS. 2 and 3;

FIG. 5 is a flow diagram of a method for administering nutritional products according to the invention.

### DETAILED DESCRIPTION

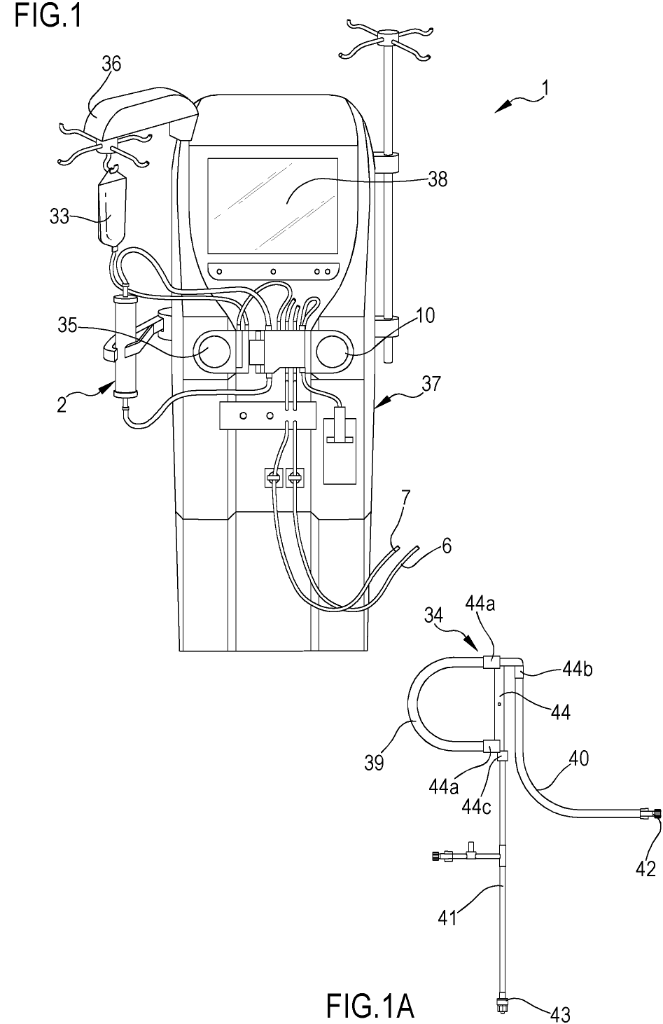

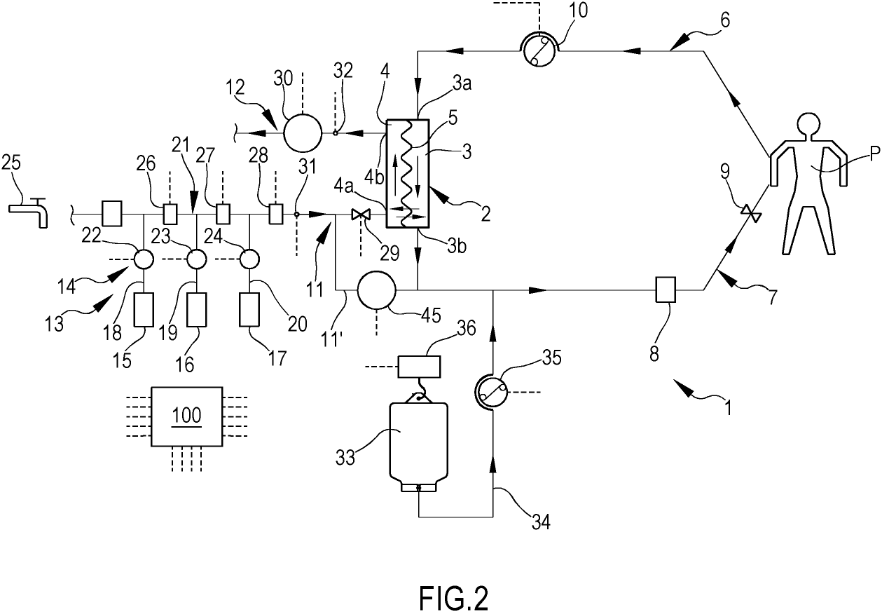

An apparatus 1 for extracorporeal blood treatment for chronic (long term) therapy is represented in FIGS. 1 and 2. The apparatus 1 comprises a filtration unit 2 having a primary chamber 3 and a secondary chamber 4 separated by a semi-permeable membrane 5. Depending upon the treatment, the semi-permeable membrane 5 of the filtration unit 2 may be selected to have different properties and performances.

A blood circuit is coupled to the primary chamber 3 of the filtration unit 2. The blood circuit comprises a blood withdrawal line 6 connected to an inlet 3a of the primary chamber 3, a blood return line 7 connected to an outlet 3b of the primary chamber 3. The withdrawal line 6 and blood return line 7 are configured for connection to a cardiovascular system of a patient “P”.

In use, the blood withdrawal line 6 and the blood return line 7 are connected to a needle or to a catheter or other access device which is then placed in fluid communication with the patient “P” vascular system, such that blood may be withdrawn through the blood withdrawal line 6, flown through the primary chamber 3 and then returned to the patient’s vascular system through the blood return line 7. An air separator, such as a deaeration chamber 8, may be present on the blood return line 7. Moreover, a monitor valve 9 may be present on the blood return line 7, downstream the deaeration chamber 8.

The blood flow through the blood circuit is controlled by a blood pump 10, for instance a peristaltic blood pump, acting either on the blood withdrawal line 6 or on the blood return line 7. The embodiment of FIG. 2 shows the blood pump 10 coupled to a pump section of the withdrawal line 6. A control unit 100 is connected and controls the blood pump 10 to regulate a blood flow rate.

A dialysis circuit is connected to the secondary chamber 4 of the filtration unit 2 and comprises a dialysis supply line 11 connected to an inlet 4a of the secondary chamber 4 and a dialysis effluent line 12 connected to an outlet 4b of the secondary chamber 4 and to a drain, not shown.

The dialysis supply line 11 is connected to a preparation device 13 for preparing a fresh dialysis fluid. The preparation device 13 comprises a regulating device 14 for regulating the composition of the fresh dialysis fluid. The dialysis effluent line discharges a spent dialysis fluid into the drain.

In the example of FIG. 2, the preparation device 13 comprises three containers of concentrate 15, 16, 17 located on respective injection lines 18, 19, 20 which are predisposed to supply substances such as electrolytes, buffer agents or others towards a preparation line 21 of the fresh dialysis fluid. The concentrate containers 15, 16, 17 may comprise concentrates in the liquid state or solid state, for example powder.

The regulating device 14 comprises injection pumps 22, 23, 24 placed on the injection lines 18, 19, 20 to move the fluid along the respective injection line 18, 19, 20 towards the preparation line 21 which collects the liquid, for example water, from a source 25. The preparation line 21 is located upstream the dialysis supply line 11 and has one end connected to the source 25, e.g. a deionized/purified water source or a reverse osmosis water plant, and an opposite end connected to the dialysis supply line 11. The source 25 may comprise the water source, as shown, or a source of ultra-pure liquid.

Concentration or conductivity sensors 26, 27, 28 are located on the preparation line 21 and are able to provide the control unit 100 with a signal related to conductivity or concentration of a predetermined substance (for example sodium) of the fluid crossing the preparation line 21 such that the control unit 100 is able to control the injection pumps 22, 23, 24 in order to regulate the conductivity Cd or concentration, for example of sodium [Na], of the liquid crossing the dialysis supply line 11. A fluid check organ 29 may be used to selectively enable or inhibit a passage of fluid across the dialysis line 21 and into the filtration unit 2 in case the liquid does not meet the required parameters.

An infusion line 11′ departs from the dialysis supply line 11 and is connected to the blood return line 7 to infuse part of the fresh dialysis fluid into the blood circuit.

An auxiliary infusion pump 45 may be coupled to the infusion line 11′ to deliver said part of the fresh dialysis fluid into the blood circuit.

Of course the infusion line 11′ may alternatively or in addition being connected to the blood withdrawal line 6 (in particular downstream the blood pump 10) for pre-infusing a substitution fluid.

An ultrafiltration device is configured to achieve a fluid removal from the patient through the semi-permeable membrane 5 of the filtration unit 2. The ultrafiltration device comprises a dialysis pump 30 located on the dialysis effluent line 12. In a variant embodiment, a first dialysis pump is coupled to the dialysis supply line 11 and a second dialysis pump coupled to the dialysis effluent line 12. A first flow-meter 31 is active on the dialysis supply line 11 and is placed between the fluid check organ 29 and the inlet 4a of the secondary chamber 4. A second flow-meter 32 is active on the dialysis effluent line 12 and is placed between the outlet 4a of the secondary chamber 4 and the dialysis pump 30.

The infusion line 11′ is connected to the dialysis supply line 11 between the first flow-meter 31 and the inlet 4a of the secondary chamber 4. The first flow-meter 31 and the second flow-meter 32 are connected to the control unit 100 and are configured to determine an ultrafiltration rate UFR.

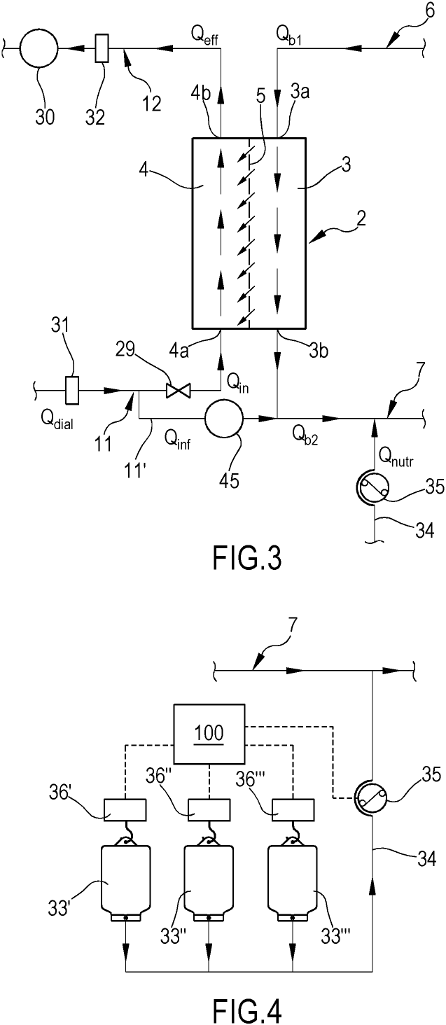

The ultrafiltration rate UFR is a difference between the spent dialysis fluid exiting the outlet 4b of the secondary chamber 4 and the prepared fresh dialysis fluid routed to the inlet 4a of the secondary chamber 4 and infused into the blood circuit through the infusion line 11′ (FIG. 3).

As shown in FIG. 3, Q_{b1} is the blood flow rate entering the primary chamber 3 through the inlet 3a, Q_{b2} is the blood flow rate leaving the primary chamber 3 through the outlet 3b, Qᵢₙ is the flow rate entering the secondary chamber 4 through the inlet 4a, Q_{inf} is the flow rate crossing the infusion line 11′, wherein:

$${\text{Q}}_{\text{dial}} = {\text{Q}}_{\text{in}} + {\text{Q}}_{\text{inf}}$$

The first flow-meter 31 and the second flow-meter 32 provide the control unit 100 with an instant value of the respective flows and thus enable the control unit 100 to calculate an instant ultrafiltration rate UFR. Alternatively, a differential sensor may be provided, active on the dialysis supply line 11 and on the dialysis effluent line 12 and therefore able directly to provide a signal relating to the ultrafiltration rate UFR. Instead of flowmeters, balance chambers may operatively be coupled to the dialysis circuit is provided. The balance chambers principle operates so that the amount of fluid entering into the first chamber on the dialysis supply line 11 is equal to the amount of fluid exiting from the dialysis effluent line 12. To achieve the ultrafiltration, an ultrafiltration line (not represented) is added to the effluent line upstream the balancing chamber. An ultrafiltration pump removes the desired amount of ultrafiltered liquid before that the spent dialysis liquid reaches the second balance chamber thereby achieving an ultrafiltered volume. In this alternative embodiment, the second parameter is related to the amount of liquid volume that is removed through the ultrafiltration line and in particular is the ultrafiltration rate through the ultrafiltration line. Of course absolute volume variation through ultrafiltration line may be measured too.

The apparatus 1 further comprises one nutritional bag 33 containing a nutritional solution and a nutritional line 34 having a first end in fluid communication with the nutritional bag 33 and a second end connected to the blood return line 7 for infusing the nutritional solution into the patient vascular system through said blood return line 7. In an alternative embodiment, the nutritional solution may be infused directly into the patient vascular system. An infusion pump 35 is coupled to the nutritional line 34 to deliver the nutritional solution through the nutritional line 34. A sensing element in the form of a weighing device 36 is configured to weigh the nutritional bag 33 while the nutritional solution is infused and to provide a first signal allowing calculation of a first parameter W related to a weight of the nutritional bag 33 and, therefore, to the weight or volume of nutritional solution contained in the nutritional bag 33. In general, the weighing device 36 provides the time variation of the bag weight which is directly linked to the actual flow rate of the nutritional solution through the nutritional line. Indeed, the control unit knows the nutritional line cross section and may therefore easily and very accurately calculate the actual flow rate of the nutritional solution through the nutritional line starting from measurements of weight/weight variation over time of the corresponding bag. Optionally the first parameter is the weight of the nutritional bag 33 measured over time. As an alternative, a flow meter on the nutritional line may be used as a sensing element.

Finally, though less precise, the pump speed may be used to determine flow rate through the nutritional line, the speed being monitored through a suitable sensor, such as a Hall sensor or a sensor sensitive to pump electric parameters such as resistance or power consumption.

As shown in FIG. 1, the apparatus 1 comprises a main body 37 provided with a base resting on the ground and hosting all the components of said apparatus 1, i.e.: the filtration unit 2, the blood circuit, the blood pump 10, the dialysis circuit, the preparation device 13, the weighing device 36, the nutritional line 34, the infusion pump 35, the control unit 100. The weighing device 36 is a weight scale and is configured to hang the nutritional bag 33.

The control unit 100 is housed in the main body 37. The infusion pump 35 and the blood pump 10 are peristaltic pumps supported by the main body 37. Each peristaltic pump comprises an actuator or motor, not shown, connected to a rotor. The rotor of the infusion pump 35 and the rotor of the blood pump 10 are placed on a front face of the main body 37.

The blood circuit and the filtration unit 2 are disposable (i.e. they are disposed after each blood treatment) and are coupled in removable manner to the main body 37. The pump section of the withdrawal line 6 is coupled in removable manner to the rotor of the blood pump 10. The nutritional line 34 is disposable and is coupled in removable manner to the main body 37. A pump section of the nutritional line 34 is coupled in removable manner to the infusion pump 35.

FIG. 1A shows an example of disposable nutritional line 34. The nutritional line 34 comprises a pump segment 39, a feeding tube segment 40 connected to an inlet of the pump segment 39, and a delivery tube segment 41 connected to an outlet of the pump segment 39. In particular, the pump segment 39 may have a passage section for the nutritional solution larger than a passage section of the delivery tube segment 41 and/or than a passage section of the feeding tube segment 40. In general, the passage section of the delivery tube segment is equal to the passage section of the feeding tube segment. On a first end for connection to the nutritional bag (or to a tubing connected to the nutritional bag), the feeding tube segment 40 includes a respective removable connector 42, in particular a Luer connector. The other end is joined to a rigid connector 44b associated to a rigid portion 44 of the nutritional line 34 supporting the pump segment 39. Correspondingly, the delivery tube segment 41 includes a respective removable connector 43, in particular a Luer connector, connectable to a corresponding counter connector on the blood return line 7, for example at the deareation chamber 8. As mentioned, the nutritional line further comprises one rigid portion 44 (the portion being more rigid than the flexible feeding and delivery tube segments 40, 41) comprising two pump connectors 44a for receiving opposite ends of the pump segment, the delivery tube segment connector 44c for receiving one end of the delivery tube segment 41 and the feeding tube segment connector 44b for receiving one end of the feeding tube segment 40.

The extracorporeal blood treatment apparatus main body 37 includes a coupling device; the rigid body 44 is configured to couple with the apparatus coupling device to position the nutritional line on the main body in specific arrangement with respect to the infusion pump so that the pump segment may be precisely received around the infusion pump rotor. In particular the coupling device is placed on the front panel of the main body 37.

The dialysis circuit is not-disposable and is configured to be sterilized after each blood treatment. Tubes, pumps, sensors of the dialysate circuit are not configured to be replaced after each treatment but only for maintenance purposes or in case of faults. Therefore, the dialysis circuit is integrated in the main body 36 and mounted fixed in or on the main body 37. The dialysis pump 30 is a volumetric pump crossed by the effluent fluid and is mounted in the main body 37.

The control unit 100 is contained in or supported by the main body 37. The control unit 100 is connected to the blood pump 10, the weighing device 36, the infusion pump 35, the injection pumps 22, 23, 24 of the regulating device 14, the concentration or conductivity sensors 26, 27, 28, the first flow-meter 31, the second flow-meter 32, the fluid check organ 29 and to the dialysis pump 30 of the ultrafiltration device. The control unit 100 controls the weighing device to measure the weight at instants of time, e.g. with an acquisition frequency 1 Hertz. The control unit 100 is also connected to a display screen 38 (FIG. 1) mounted on the main body 37.

The control unit 100 may comprise a digital processor (CPU) with memory (or memories), an analogical type circuit, or a combination of one or more digital processing units with one or more analogical processing circuits. In the present description and in the claims it is indicated that the control unit 100 is “configured” or “programmed” to execute steps: this may be achieved in practice by any means which allow configuring or programming the control unit 100. For instance, in case of a control unit 100 comprising one or more CPUs, one or more programs are stored in an appropriate memory: the program or programs containing instructions which, when executed by the control unit 100, cause the control unit 100 to execute the steps described and/or claimed in connection with the control unit 100. Alternatively, if the control unit 100 is of an analogical type, then the circuitry of the control unit 100 is designed to include circuitry configured, in use, to process electric signals such as to execute the control unit 100 steps herein disclosed.

The control unit 100 is configured to or programmed for receiving signals from the sensors and from other inputs and for commanding pumps and valves according to said signals, in order to perform the blood treatment and to administer nutritional products to the patient during blood treatment. The control unit 100 is also configured to or programmed for displaying data on the display screen 38.

The nutritional solution contained in the nutritional bag 33 comprises a mixture of amino-acids, glucose or dextrose and lipids. For instance, the solution consists of 300 ml of amino-acids at a concentration of 15%, 150 ml of dextrose at a concentration of 50% and 150 ml of lipids at a concentration of 20%. The amount of nutritional solution contained in the nutritional bag 33 may be the total amount administered to the patient at the end of the blood treatment and may correspond to 800 kcal per treatment or 200 kcal/h.

In an alternative embodiment, shown in FIG. 4, a plurality of nutritional bags 33′, 33″, 33‴ are provided and each nutritional bag containing a component of the nutritional solution. A first bag 33′ contains a solution of amino-acids, a second bag 33″ contains a solution of glucose or dextrose, a third bag 33‴ contains a solution of lipids. The three nutritional bags 33′, 33″, 33‴ are each hanging on a respective weight scale 36′, 36″, 36‴ and connected to a common nutritional line 34 provided with the infusion pump 35. In an alternative embodiment, not shown, each of the three nutritional bags 33′, 33″, 33‴ may be coupled to a respective infusion pump.

According also to the method of the invention, the control unit 100 is programmed for receiving a patient prescription for the blood treatment, i.e. target values for the blood treatment and target values for the administration of the nutritional products to the patient. These target values may be entered manually, e.g. through the display touch screen 38 or a keyboard placed on the main body 37 of the apparatus 1. The prescription may alternatively be read from a patient card or from another kind of storage media device or received by the machine by means of a data transmission channel (wired or wireless).

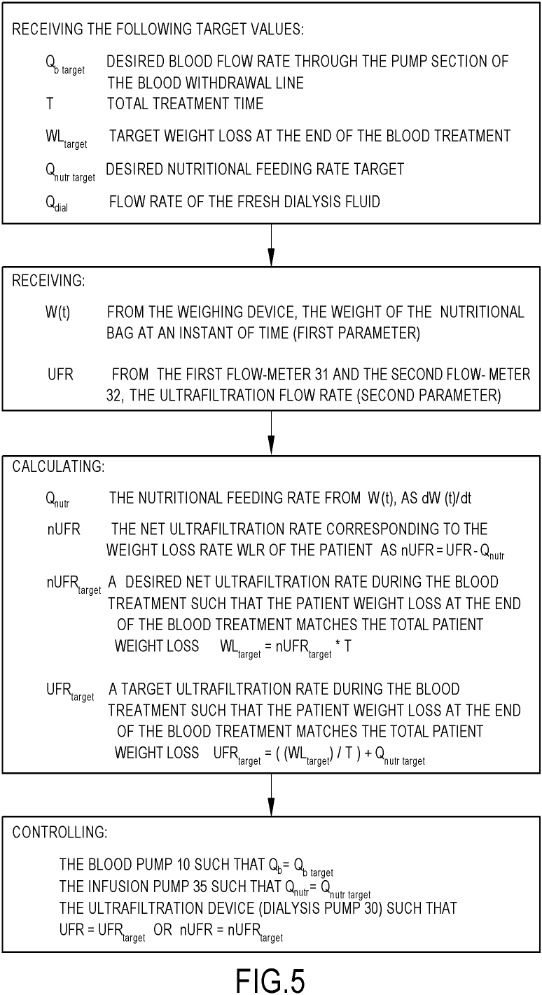

Example (FIG. 5 ) The control unit 100 is programmed for receiving as input the following target values:

| | |
|---|---|
| Q_{b} _{target} | Desired blood flow rate through the pump section of the blood withdrawal line |
| T | Total treatment time |
| WL_{target} | Target weight loss at the end of the blood treatment |
| Qₙᵤₜᵣ _{target} | Desired nutritional feeding rate target |
| Q_{dial} | Flow rate of the fresh dialysis fluid |

The control unit 100 is programmed for receiving:

| | |
|---|---|
| W(t) | from the weighing device, the weight of the nutritional bag at an instant of time (first parameter) |

| | |
|---|---|
| UFR | from the first flow-meter 31 and the second flow-meter 32, the ultrafiltration flow rate (second parameter) |

The control unit 100 is programmed for calculating:

| | |
|---|---|
| Qₙᵤₜᵣ | the nutritional feeding rate from W(t), as dW(t)/dt |
| nUFR | the net ultrafiltration rate corresponding to the weight loss rate WLR of the patient as nUFR = |
| | UFR - Qₙᵤₜᵣ |
| nUFR_{target} | a desired net ultrafiltration rate during the blood treatment such that the patient weight loss at the end of the blood treatment matches the total patient weight loss |
| | ${\text{WL}}_{\text{target}} = {\text{nUFR}}_{\text{target}} \text{*} \text{T}$ |
| UFR_{target} | a target ultrafiltration rate during the blood treatment such that the patient weight loss at the end of the blood treatment matches the total patient weight loss WLR*T = nUFR*T = WL(T) = WL_{target} |
| | ${\text{WL}}_{\text{target}} = {\text{nUFR}}_{\text{target}} \text{*} \text{T} = {\text{UFR}}_{\text{target}} \text{*} \text{T} \text{-} {\text{Q}}_{\text{nutr} \text{target}} \text{*} \text{T}$ |
| | ${\text{UFR}}_{\text{target}} = \left(\left({\text{WL}}_{\text{target}}\right) / \text{T}\right) + {\text{Q}}_{\text{nutr} \text{target}}$ |

The control unit 100 is programmed for controlling:
the blood pump 10 such that Q_{b} = Q_{btarget}
the infusion pump 35 such that Qₙᵤₜᵣ = Q_{nutrtarget}
the ultrafiltration device (dialysis pump 30) such that UFR = UFR_{target} or nUFR = nUFR_{target}

In particular, the control unit 100 is programmed for controlling the ultrafiltration device (dialysis pump 30) such that the flow rate of the spent dialysis fluid Q_{eff} is equal to Q_{eff} _{target} wherein

$${\text{Q}}_{\text{eff} \text{target}} = {\text{UFR}}_{\text{target}} + {\text{Q}}_{\text{dial}} = \left(\left({\text{WL}}_{\text{target}}\right) / \text{T}\right) + {\text{Q}}_{\text{nutr} \text{target}} + {\text{Q}}_{\text{dial}}$$

Numerical example

| | |
|---|---|
| T | 4 hours |
| WL_{target} | 3.5 kg = 3500 ml (blood density is very close to water density) |
| Qₙᵤₜᵣ _{target} | 150 ml/hour = 2.5 ml/min |
| Q_{dial} | 500 ml/min |

$${\text{Q}}_{\text{eff} \text{target}} = \left(\left({\text{WL}}_{\text{target}}\right) / \text{T}\right) + {\text{Q}}_{\text{nutr} \text{target}} + {\text{Q}}_{\text{dial}} = 517 \text{ml} / \text{min}$$

According to other embodiments, the target value entered directly into the control unit 100 may be, instead of the target weight loss rate WLR_{target}, the desired net ultrafiltration rate nUFR_{target} (corresponding to the target weight loss rate WLR_{target}).

According to other embodiments, instead of the desired nutritional feeding rate target Qₙᵤₜᵣ _{target}, the control unit 100 is programmed for receiving as input a total amount of the nutritional solution W_{nutr target} administered to the patient P at the end of the treatment time T. The control unit 100 is programmed for calculating the desired nutritional feeding rate target Qₙᵤₜᵣ _{target} as Wₙᵤₜᵣ _{target}/T or to calculate a desired nutritional feeding rate target Qₙᵤₜᵣ _{target}(t) which changes over time during treatment and such that the integration of Qₙᵤₜᵣ _{target}(t) over time T is equal to Wₙᵤₜᵣ _{target}.

According to other embodiments, the control unit is programmed for integrating the ultrafiltration flow rate UFR (second parameter) received from the first flow-meter 31 and the second flow-meter 32, and by subtracting the weight W(t) of the nutritional bag (first parameter) from the integrated ultrafiltration rate UFR to calculate the patient weight loss (WL(t)) at time t. Then to compare the calculated patient weight loss (WL(t)) at time t with the target total weight loss at the end of the blood treatment WL_{target} and to stop the treatment when the patient weight loss is equal to WL_{target}.

According to some embodiments, the nutritional feeding rate Qₙᵤₜᵣ, the ultrafiltration flow rate UFR(t), the net ultrafiltration rate nUFR(t) and other related values are constant over time during blood treatment.

According to some embodiments, the feeding rate Qₙᵤₜᵣ of the nutritional solution, the ultrafiltration flow rate UFR(t), the net ultrafiltration rate nUFR(t) and other related values may be controlled to change over time during blood treatment. For instance, the net ultrafiltration rate nUFR(t) (or the weight loss rate WLR) may be greater at the start of the blood treatment, when the patient P may release more of liquid, than at the end. For instance, the nutritional feeding rate target Qₙᵤₜᵣ may be greater at the end of the blood treatment, where it is more likely that the nutritional solution is not eliminated through the blood treatment, than at the start.

The control unit 100 may be programmed for changing the feeding rate Qₙᵤₜᵣ of the nutritional solution by controlling the infusion pump 35 and/or for changing the ultrafiltration flow rate UFR(t) by controlling the dialysis pump 30.

The control unit 100 may also be programmed for storing data related to the nutritional solution administered during the extracorporeal blood treatment and to display on the display screen said data together with other values related to the blood treatment. For instance, said data may comprise at least one of: the weight of the at least one nutritional bag, a feeding rate Qₙᵤₜᵣ, an amount of nutritional solution administered at an instant of time, the total amount of the nutritional solution to be administered, a composition of the nutritional solution.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the scope of the appended claims.

1-33. (canceled)

34. An apparatus for extracorporeal blood treatment for chronic therapy, the apparatus comprising:
a filtration unit having a primary chamber and a secondary chamber separated by a semi-permeable membrane;
a blood circuit coupled to the filtration unit and comprising a blood withdrawal line connected to an inlet of the primary chamber, a blood return line connected to an outlet of the primary chamber, said blood withdrawal line and blood return line being configured for connection to a patient cardiovascular system;
a blood pump configured to be coupled to a pump section of the blood circuit;
a dialysis circuit comprising a dialysis supply line for a fresh dialysis fluid connected to an inlet of the secondary chamber and a dialysis effluent line for a spent dialysis fluid connected to an outlet of the secondary chamber;
a preparation device for preparing the fresh dialysis fluid, wherein the preparation device is connected to the dialysis supply line and comprises a regulating device for regulating the composition of the fresh dialysis fluid;
a nutritional bag containing a nutritional solution;
a nutritional line having a first end in fluid communication with the nutritional bag and a second end for infusing the nutritional solution into either the blood return line or directly into the patient vascular system;
at least one sensing element configured to provide a first signal related to an actual flow rate in the nutritional line;
an ultrafiltration device configured to achieve a fluid removal from the patient through the semi-permeable membrane;
at least one sensor configured to provide a second signal related to an ultrafiltration rate; and
a control unit connected to the ultrafiltration device, to the sensing element, and to the at least one sensor, the control unit programmed for:
receiving a patient prescription including at least one of:
a total patient weight loss to be achieved at the end of the blood treatment and a total treatment time, and
a desired net ultrafiltration rate,
collecting from the sensing element the first signal and determining a first parameter related to the actual flow rate in the nutritional line,
collecting from the at least one sensor the second signal and determining a second parameter related to the ultrafiltration rate, and
controlling the ultrafiltration device to achieve the patient prescription based on the first parameter and on the second parameter.

35. The apparatus of claim 34, wherein the at least one sensing element is a weighing device configured to provide a weight of the nutritional bag, wherein the control unit is configured to receive a weight signal from the weighing device and to determine the first parameter based on the weight variation over time of the nutritional bag, the first parameter being the actual flow rate in the nutritional line.

36. The apparatus of claim 34, wherein the control unit is programmed for calculating and/or storing data related to the nutritional solution administered during the extracorporeal blood treatment, said data comprising at least one of: the weight of the nutritional bag, a feeding rate of the nutritional solution through the nutritional line, an amount of nutritional solution administered at an instant of time, a total amount of the nutritional solution to be administered, or a composition of the nutritional solution.

37. The apparatus of claim 34, comprising the nutritional solution contained in said nutritional bag, said nutritional solution comprising a mixture of protein, carbohydrate, and fat.

38. The apparatus of claim 34, wherein a feeding rate of the nutritional solution during blood treatment is between 50 ml/h and 500 ml/h.

39. The apparatus of claim 34, comprising an infusion pump coupled to the nutritional line to deliver a feeding rate of the nutritional solution through the nutritional line, wherein the control unit is connected to the infusion pump and is programmed for controlling the infusion pump and for changing the feeding rate of the nutritional solution.

40. The apparatus of claim 34, wherein the first parameter includes the weight of the nutritional bag at an instant of time, and wherein the second parameter includes the ultrafiltration rate.

41. The apparatus of the preceding claim 40, wherein a net ultrafiltration rate is calculated by calculating a feeding rate of the nutritional solution from the weight of the nutritional bag and by subtracting the feeding rate from the ultrafiltration rate, and wherein the ultrafiltration device is controlled so that the net ultrafiltration rate matches the desired net ultrafiltration rate.

42. The apparatus of the preceding claim 40, wherein a patient weight loss at an instant of time is calculated by integrating the ultrafiltration rate at that instant of time and by subtracting the weight of the nutritional bag at that instant of time from the integrated flow rate, and wherein the ultrafiltration device is controlled so that the patient weight loss at the end of the blood treatment matches the total patient weight loss.

43. The apparatus of claim 34, wherein the at least one sensor comprises a flowmeter placed on at least one of the dialysis supply line and the dialysis effluent line, and wherein the second signal is a signal from said flowmeter.

44. The apparatus of claim 34, wherein the nutritional line comprises a pump segment, a feeding tube segment connected to an inlet of the pump segment, a delivery tube segment connected to an outlet of the pump segment and one rigid portion comprising two pump connectors for receiving opposite ends of the pump segment, a delivery tube segment connector for receiving one end of the delivery tube segment, and a feeding tube segment connector for receiving one end of the feeding tube segment,
wherein the feeding tube segment and/or the delivery tube segment includes a respective removable connector, the removable connector of the delivery tube segment configured to be connected to a corresponding counter connector on the blood return line.

45. The apparatus of claim 36, comprising a display screen connected to the control unit, wherein the control unit is programmed for displaying on said display screen the data related to the nutritional solution.

46. The apparatus of claim 39, comprising a main body, wherein the control unit is contained in or supported by the main body, wherein the infusion pump and the blood pump are supported by the main body, wherein the blood circuit and the filtration unit are disposable and are coupled in removable manner to the main body and to the blood pump, wherein the nutritional line is disposable and is coupled in removable manner to the main body and to the infusion pump, and wherein the dialysis circuit is non-disposable and is integrated in the main body.

47. The apparatus of claim 46, wherein the infusion pump and the blood pump are peristaltic pumps.

48. The apparatus of claim 46, wherein the ultrafiltration device comprises at least one dialysis pump coupled to the dialysis supply line and/or to the dialysis effluent line, and wherein said at least one dialysis pump is mounted on the main body.

49. The apparatus of claim 34, wherein the patient prescription includes at least one of:
(i) a total patient weight loss to be achieved at the end of the blood treatment and a total treatment time,
(ii) an ultrafiltration rate and a total treatment time,
(iii) a total patient weight loss to be achieved at the end of the blood treatment and an ultrafiltration rate, and
(iv) a desired net ultrafiltration rate and a nutritional flow rate, or a nutritional total weight to be infused during the treatment, together with a total treatment time or together with a total patient weight loss.

50. The apparatus of claim 34, wherein the sensor for providing the second signal related to an ultrafiltration rate senses one or more of:
a difference between an amount or a flow rate of fresh dialysis fluid prepared by the preparation device and entering into the filtration unit and a spent dialysis fluid exiting the filtration unit;
a difference between an amount or a flow rate of fresh dialysis fluid prepared by the preparation device and infused into the blood circuit and a spent dialysis fluid exiting the filtration unit; and
a difference between an amount or a flow rate of fresh dialysis fluid prepared by the preparation device and entering both into the filtration unit and into the blood circuit and a spent dialysis fluid exiting the filtration unit.

51. The apparatus of claim 34, wherein the sensor for providing the second signal related to an ultrafiltration rate takes into account a net amount/flow rate of the fresh dialysis fluid prepared by the preparation device, irrespective of the fresh dialysis fluid being fed to the filtration unit, removed from the filtration unit and infused into the blood circuit.

52. The apparatus of claim 34, wherein the apparatus comprises an infusion line branching from the dialysis supply line to infuse fresh dialysis fluid into the blood circuit, the sensor configured to provide a second signal related to an ultrafiltration rate being operative at least upstream a branch of the infusion line to take into account for the fresh dialysis fluid fed either or both to the filtration unit through the dialysis supply line or/and the blood circuit through the infusion line.

53. The apparatus of claim 34, wherein the at least one sensor comprises either:
a flowmeter placed on the dialysis supply line and another flowmeter placed on the dialysis effluent line, the control unit configured to receive the signals from the two flowmeters to determine a differential flow between dialysis fluid supplied to the supply line and removed with the dialysis effluent line; or
a differential flowmeter placed on the dialysis supply line and the dialysis effluent line to sense a differential flow between the dialysis supply line and the dialysis effluent line.

54. The apparatus of claim 34, further comprising:
balance chambers operatively coupled to the dialysis circuit to precisely balance fresh dialysis fluid prepared by the preparation device with spent dialysis fluid exiting the filtration unit; and
an ultrafiltration line and pump to remove spent dialysis fluid from upstream of the balance chamber in the dialysis effluent line, the at least one sensor sensing the amount of spent dialysis fluid removed by the ultrafiltration pump, wherein the second parameter is a flow rate in the ultrafiltration line or a liquid volume removed through the ultrafiltration line.

55. The apparatus of claim 54, wherein the ultrafiltration device comprises a first dialysis pump coupled to the dialysis supply line and a second dialysis pump coupled to the dialysis effluent line.

56. The apparatus of claim 54, wherein a first balance chamber operates on the dialysis supply line and a second balance chamber operates on the dialysis effluent line.

57. The apparatus of claim 34, wherein the control unit is programmed for: receiving, as prescription input:
a desired blood flow rate,
a desired nutritional feeding rate target or a total amount of the nutritional solution administered at the end of the treatment time, and
the total patient weight loss and the total treatment time or the desired net ultrafiltration rate,
commanding the blood pump to pump blood according to the desired blood flow rate or to reach the total amount of the nutritional solution administered at the end of the total treatment time,
commanding the infusion pump to deliver the nutritional solution according to the nutritional feeding rate target, and
commanding the ultrafiltration device to achieve the total patient weight loss or the desired net ultrafiltration rate.

58. The apparatus of claim 34, wherein the control unit is programmed to receive, as a prescription input, a total amount of the nutritional solution to be administered within the end of the total treatment time, to determine a nutritional feeding rate target to deliver the total amount of the nutritional solution at the latest at an end of the total treatment time and to command the infusion pump to deliver the nutritional solution according to the nutritional feeding rate target.

59. The apparatus of claim 34, wherein the extracorporeal blood treatment apparatus includes a main body, and wherein the infusion pump is attached to the main body and is placed on a front panel of the main body.

60. An apparatus for extracorporeal blood treatment for chronic therapy comprising:
a main body;
a filtration unit having a primary chamber and a secondary chamber separated by a semi-permeable membrane;
a blood circuit coupled to the filtration unit and comprising a blood withdrawal line connected to an inlet of the primary chamber, a blood return line connected to an outlet of the primary chamber, said blood withdrawal line and blood return line being configured for connection to a patient cardiovascular system;
a blood pump configured to be coupled to a pump section of the blood circuit;
a dialysis circuit comprising a dialysis supply line, for a fresh dialysis fluid, connected to an inlet of the secondary chamber and a dialysis effluent line, for a spent dialysis fluid, connected to an outlet of the secondary chamber;
a preparation device for preparing the fresh dialysis fluid comprising a preparation line connected to a liquid source and a container of concentrate, wherein the container is located on a respective injection line predisposed to supply substances to the preparation line, wherein the preparation device is connected to the dialysis supply line and comprises a regulating device for regulating the composition of the fresh dialysis fluid;
a nutritional solution comprising a mixture of protein, carbohydrate and fat;
a nutritional bag containing the nutritional solution;
a nutritional line having a first end in fluid communication with the nutritional bag and a second end for infusing the nutritional solution into either the blood return line or directly into the patient vascular system;
an infusion pump attached to the main body and coupled to the nutritional line to deliver a feeding rate of the nutritional solution through the nutritional line;
at least one sensing element, including a weighing device configured to provide a weight of the at least one nutritional bag, configured to provide a first signal related to an actual flow rate in the nutritional line;
an ultrafiltration device configured to achieve a fluid removal from the patient through the semi-permeable membrane;
at least one sensor configured to provide a second signal related to an ultrafiltration rate and sensing one of:
a difference between an amount or a flow rate of fresh dialysis fluid prepared by the preparation device and entering into the filtration unit and a spent dialysis fluid exiting the filtration unit,
a difference between an amount or a flow rate of fresh dialysis fluid prepared by the preparation device and infused into the blood circuit and a spent dialysis fluid exiting the filtration unit, and
a difference between an amount or a flow rate of fresh dialysis fluid prepared by the preparation device and entering both into the filtration unit and into the blood circuit and a spent dialysis fluid exiting the filtration unit; and
a control unit connected to the ultrafiltration device, to the infusion pump, to the sensing element and to the at least one sensor, the control unit programmed for:
receiving a patient prescription including two parameters out of:
a total patient weight loss to be achieved at the end of the blood treatment,
a total treatment time, and
the ultrafiltration rate,
receiving a weight signal from the weighing device and determining the first parameter being the actual flow rate in the nutritional line,
collecting from the at least one sensor the second signal and determining a second parameter being the ultrafiltration rate,
controlling the infusion pump, and
controlling the ultrafiltration device to achieve the patient prescription based on the first parameter and on the second parameter so that the patient weight loss at the end of the blood treatment matches the total patient weight loss to be achieved at the end of the blood treatment.

61. The apparatus of claim 60, comprising an infusion line branching from the dialysis supply line to infuse fresh dialysis fluid into the blood circuit, the sensor configured to provide a second signal related to an ultrafiltration rate being operative upstream of a branch of the infusion line to take into account for the fresh dialysis fluid fed either or both to the filtration unit through the dialysis supply line or/and the blood circuit through the infusion line.

62. The apparatus of claim 60, wherein the at least one sensor comprises a differential flowmeter placed on the dialysis supply line and the dialysis effluent line to sense a differential flow between the dialysis supply line and the dialysis effluent line, and wherein the second parameter is a signal from said differential flowmeter.

63. The apparatus of claim 60, wherein the at least one sensor comprises a flowmeter placed on the dialysis supply line and another flowmeter placed on the dialysis effluent line, the control unit configured to receive the signals from the flowmeter and the another flowmeter to determine a differential flow between dialysis fluid supplied to the supply line and removed with the dialysis effluent line.

64. The apparatus of claim 63, wherein the flowmeter on the dialysis supply line is operative upstream of a branch of the infusion line to take into account for the fresh dialysis fluid fed either or both to the filtration unit through the dialysis supply line or/and the blood circuit through the infusion line.

65. The apparatus of claim 60, comprising:
balance chambers operatively coupled to the dialysis circuit to precisely balance fresh dialysis fluid prepared by the preparation device with spent dialysis fluid exiting the filtration unit; and
an ultrafiltration line and pump to remove spent dialysis fluid from upstream the balance chamber in the dialysis effluent line, the at least one sensor sensing the amount of spent dialysis fluid removed by the ultrafiltration pump, wherein the second parameter is a flow rate in the ultrafiltration line or a liquid volume removed through the ultrafiltration line.

66. The apparatus of claim 65, wherein a first balance chamber of the balance chambers operates on the dialysis supply line and a second balance chamber of the balance chambers operates on the dialysis effluent line, the first balance chamber located upstream of a branch of the infusion line to take into account for the fresh dialysis fluid fed either or both to the filtration unit through the dialysis supply line or/and the blood circuit through the infusion line.

67. An apparatus for extracorporeal blood treatment for chronic therapy comprising:
a main body;
a filtration unit having a primary chamber and a secondary chamber separated by a semi-permeable membrane;
a blood circuit coupled to the filtration unit and comprising a blood withdrawal line connected to an inlet of the primary chamber, a blood return line connected to an outlet of the primary chamber, said blood withdrawal line and blood return line configured for connection to a patient cardiovascular system;
a blood pump configured to be coupled to a pump section of the blood circuit;
a dialysis circuit comprising a dialysis supply line, for a fresh dialysis fluid, connected to an inlet of the secondary chamber and a dialysis effluent line, for a spent dialysis fluid, connected to an outlet of the secondary chamber;
a preparation device for preparing the fresh dialysis fluid comprising a preparation line connected to a liquid source and a container of concentrate, wherein the container is located on a respective injection line predisposed to supply substances to the preparation line, and wherein the preparation device is connected to the dialysis supply line and comprises a regulating device for regulating the composition of the fresh dialysis fluid;
a nutritional solution comprising a mixture of protein, carbohydrate and fat;
a nutritional bag containing the nutritional solution;
a nutritional line having a first end in fluid communication with the nutritional bag and a second end for infusing the nutritional solution into either the blood return line or directly into the patient vascular system;
an infusion pump coupled to the nutritional line to deliver a feeding rate of the nutritional solution through the nutritional line, wherein the infusion pump is attached to the main body;
at least one sensing element configured to provide a first signal related to an actual flow rate in the nutritional line;
an ultrafiltration device configured to achieve a fluid removal from the patient through the semi-permeable membrane;
at least one sensor configured to provide a second signal related to an ultrafiltration rate, the at least one sensor comprising at least one flowmeter operable with at least one of the dialysis supply line or the dialysis effluent line; and
a control unit connected to the ultrafiltration device, to the infusion pump, to the sensing element and to the at least one sensor, the control unit and programmed for:
receiving a patient prescription including at least one of:
a total patient weight loss to be achieved at the end of the blood treatment and a total treatment time, and
a desired net ultrafiltration rate,
collecting from the sensing element the first signal and determining a first parameter related to the actual flow rate in the nutritional line, wherein the at least one sensing element is a weighing device configured to provide a weight of the nutritional bag, and wherein the control unit is configured to receive a weight signal from the weighing device and to determine the first parameter based on the weight variation over time of the nutritional bag, the first parameter being the actual flow rate in the nutritional line,
collecting from the at least one sensor the second signal and determining a second parameter related to the ultrafiltration rate, wherein the second parameter is the ultrafiltration rate,
controlling the infusion pump, and
controlling the ultrafiltration device to achieve the patient prescription based on the first parameter and on the second parameter.
